(19) United States
(12) Patent Application Publication
(10) Pub. No.: US 2025/0189491 A1
(43) Pub. Date: Jun. 12, 2025

(54) MEASUREMENT APPARATUS

(71) Applicant: Fujitsu Limited, Kawasaki-shi (JP)

(72) Inventors: Suguru YASUTOMI, Kawasaki (JP); Akira SAKAI, Kawasaki (JP); Kanata SUZUKI, Kawasaki (JP)

(73) Assignee: Fujitsu Limited, Kawasaki-shi (JP)

(21) Appl. No.: 18/916,157

(22) Filed: Oct. 15, 2024

(30) Foreign Application Priority Data
Dec. 8, 2023 (JP) 2023-207818

(51) Int. Cl. G01N 29/04; G01G 21/22; G01N 29/27; G01N 33/12

(52) U.S. Cl. CPC G01N 29/04; G01G 21/22; G01N 29/27; G01N 33/12; G01N 2291/023; G01N 2291/106

(57) ABSTRACT

A measurement apparatus includes: a measurement table that has a recess inclined in a predetermined direction and an opening provided in the recess; and a processor to: discriminate whether an object is placed in the recess and is stationary; protrude a data acquisition element from the opening when discriminating that the object is placed in the recess and is stationary; press the data acquisition element against the object; and acquire measurement data on the object from the data acquisition element.

### CROSS-REFERENCE TO RELATED APPLICATION

This application is based upon and claims the benefit of priority of the prior Japanese Patent Application No. 2023-207818, filed on Dec. 8, 2023, the entire contents of which are incorporated herein by reference.

### FIELD

The embodiments discussed herein are related to a measurement apparatus.

### BACKGROUND

Quality is an important index in buying and selling frozen tuna. Since frozen tuna is hard, it is difficult to perform a same quality inspection as that for fresh fish. As a method for inspecting quality of frozen tuna, inspection using ultrasonic waves has been performed in related art. As a specific procedure, an inspector performs the inspection by holding an ultrasonic probe in his/her hand and pressing the ultrasonic probe against the frozen tuna which is a specimen.

Japanese Laid-open Patent Publication No. 2007-155692 is disclosed as related art.

### SUMMARY

According to an aspect of the embodiments, a measurement apparatus includes: a measurement table that has a recess inclined in a predetermined direction and an opening provided in the recess; and a processor to: discriminate whether an object is placed in the recess and is stationary; protrude a data acquisition element from the opening when discriminating that the object is placed in the recess and is stationary; press the data acquisition element against the object; and acquire measurement data on the object from the data acquisition element.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a schematic diagram illustrating a mechanism of a measurement apparatus;

FIG. 2 is a block diagram of the measurement apparatus;

FIG. 3 is an image diagram of a method for using the measurement apparatus;

FIG. 4 is a flowchart of measurement and quality evaluation processing by a measurement apparatus according to a first embodiment;

FIG. 5 is a block diagram of a measurement apparatus according to a first modification example;

FIG. 6 is a schematic diagram illustrating a mechanism of the measurement apparatus according to the first modification example;

FIG. 7 is a schematic perspective diagram of a measurement apparatus according to a second modification example in a state where frozen tuna is placed;

FIG. 8 is an image diagram of a measurement apparatus according to a third modification example in a state where frozen tuna is placed;

FIG. 9 is a schematic diagram of a plurality of ultrasonic probes for performing measurement of frozen tuna;

FIG. 10 is a diagram illustrating a procedure of quality evaluation by a measurement apparatus according to a third embodiment; and

FIG. 11 is a hardware configuration diagram of a measurement control device.

### DESCRIPTION OF EMBODIMENTS

For example, a technique has been proposed in which ultrasonic tomograms of tuna and numerical values of a bulk modulus, an acoustic impedance, an attenuation constant, and a Doppler shift frequency of ultrasonic tissue characteristic values are measured by using an ultrasonic inspection apparatus, and quality evaluation is performed based on the measurement results.

With the related-art quality inspection method for frozen tuna using the ultrasonic waves, a way the inspector presses the ultrasonic probe held in his/her hand against the frozen tuna varies depending on the inspector, and the way of pressing the ultrasonic probe against the tuna is not consistent. For example, with frozen tuna or the like, irregularities due to ice blocks or the like are present on a surface, and there is a large difference in the way of pressing the ultrasonic probe against the tuna depending on the inspector. Consequently, it is difficult to objectively perform consistent quality evaluation, and it is difficult to improve an accuracy of data measurement.

In view of the above, an object of the technique disclosed herein is to provide a measurement apparatus that improves an accuracy of data measurement.

Based on the drawings, embodiments of a measurement apparatus disclosed in the present application will be described in detail below. The measurement apparatus disclosed in the present application is not limited by the following embodiments.

### First Embodiment

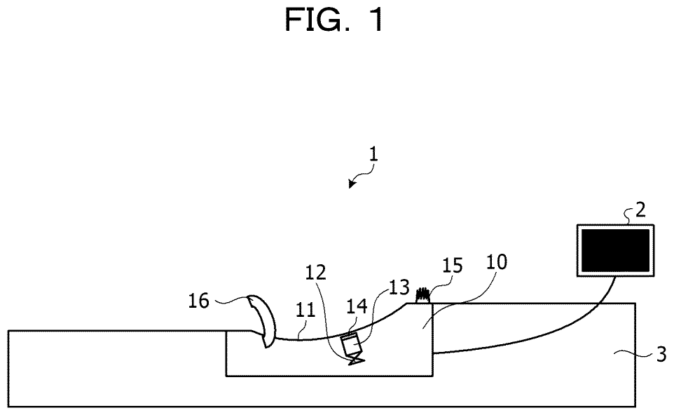

FIG. 1 is a schematic diagram illustrating a mechanism of a measurement apparatus. With frozen fish as an object to be inspected, a measurement apparatus 1 performs quality evaluation of the object by using measurement data obtained by ultrasonic measurement on the object placed over the measurement apparatus. In the following description, frozen tuna is used as an example of the object. The measurement apparatus 1 has a measurement table 10 on which frozen tuna is placed. A result display monitor 2 is coupled to the measurement apparatus 1.

As illustrated in FIG. 1, the measurement table 10 is embedded in a ground 3 having a height difference between two points such that one point is higher than the other point. The measurement table 10 may be embedded in a table having a height difference other than the ground 3. The measurement table 10 is provided with an inclined surface having an inclination from a high point to a low point in accordance with the height difference of the ground 3. The measurement table 10 is installed so as to be at substantially the same height as the ground 3 at each of the points of a high-side end and a low-side end of the inclined surface. As illustrated in FIG. 1, the measurement table 10 has a recess 11, a lifting mechanism 12, an ultrasonic probe 13, a coupling material 14, a brush 15, and a stopper 16.

The recess 11 is formed in the inclined surface of the measurement table 10. For example, the recess 11 has an inclination in which a direction from the high point to the low point is a predetermined direction in accordance with the height difference of the ground 3. The recess 11 has a shape in which frozen tuna is placed and the placed frozen tuna is fixed at a predetermined position. In the present embodiment, the recess 11 has an elliptical shape in accordance with a size of the fish as the object. When frozen albacore tuna is the object, the recess 11 may be, for example, a depression having an elliptical shape with a long axis of 70 cm and a short axis of 15 cm and having a depth of about 3 cm.

The recess 11 is formed in the inclined surface of the measurement table 10 so that the frozen tuna may be placed in a state where a head portion of the frozen tuna is positioned at a lower side of the inclined surface of the measurement table 10 and a tail fin side faces a higher side of the inclined surface of the measurement table 10. An opening is provided at a portion corresponding to a predetermined position of the placed frozen tuna in the recess 11.

The lifting mechanism 12 has a mechanism that extends and contracts. The lifting mechanism 12 is installed inside the measurement table 10, and an extension and contraction direction of the lifting mechanism 12 is a direction in which the lifting mechanism 12 passes through the opening of the recess 11. The ultrasonic probe 13 is coupled and fixed to one end of the lifting mechanism 12 on the opening side in the extension and contraction direction. The other end of the lifting mechanism 12 opposite to the ultrasonic probe 13 in the extension and contraction direction is fixed to the measurement table 10.

The ultrasonic probe 13 is a linear array probe in which an irradiation unit of ultrasonic waves and a reception unit of reflected waves are installed at the same place. By receiving designation of irradiation settings of the ultrasonic waves such as a frequency, a power, and a direction, the ultrasonic probe 13 irradiates the frozen tuna with the ultrasonic waves in accordance with the designated irradiation settings, and measures the frozen tuna by using an echo method in which the ultrasonic waves reflected by the frozen tuna are received again. The ultrasonic probe 13 corresponds to an example of a “data acquisition unit”.

The coupling material 14 is attached to a portion of the ultrasonic probe 13 that comes into contact with the frozen tuna. The coupling material 14 is formed of a flexible material having a low freezing point such as silicone rubber. Acoustic characteristics of the coupling material 14 are preferably similar to those of the object. Since the coupling material 14 has flexibility, the coupling material 14 comes into close contact with the frozen tuna by being pressed against the frozen tuna. Consequently, the coupling material 14 may come into close contact with the frozen tuna even in a case where irregularities due to ice blocks or the like are present on the surface, such as frozen tuna, and reflection from the surface of the object may be suppressed to improve an accuracy of data measurement using ultrasonic waves.

By the lifting mechanism 12 extending in a state where the frozen tuna is placed in the recess 11, the ultrasonic probe 13 with the coupling material 14 attached is lifted on the frozen tuna side and protrudes from the opening of the recess 11. When the ultrasonic probe 13 with the coupling material 14 attached is pressed against the frozen tuna, the coupling material 14 comes into close contact with the frozen tuna due to the weight of the frozen tuna.

By contraction of the lifting mechanism 12 from the state where the coupling material 14 is in close contact with the frozen tuna, the ultrasonic probe 13 is pulled back, and the coupling material 14 is separated from the frozen tuna. By further contraction of the lifting mechanism 12, the ultrasonic probe 13 with the coupling material 14 attached passes through the opening of the recess 11 and is housed inside the measurement table 10.

The brush 15 is installed in a vicinity of a high-side end portion of the inclined surface of the measurement table 10. The brush 15 is formed of, for example, a material used for a scrubbing brush. By the frozen tuna passing over the brush 15, the brush 15 removes dirt, ice blocks, and the like adhering to the frozen tuna.

The stopper 16 is installed in a vicinity of a low-side end portion of the inclined surface of the measurement table 10 in the recess 11. The stopper 16 has an annular shape having an opening in a direction orthogonal to an inclination direction of the inclined surface. In the present embodiment, the stopper 16 has, for example, a circular shape. However, the stopper 16 may have another shape, and may be a triangular or quadrangular annular shape, and a shape of an outer periphery and a shape of an inner periphery may be different from each other.

The head portion of the frozen tuna placed in the recess 11 is inserted into the stopper 16, and the stopper 16 supports and fixes the head portion so that the frozen tuna does not fall down the inclined surface. The stopper 16 has an openable and closable structure capable of separating a circular ring and releasing the supported frozen tuna.

The result display monitor 2 displays a result of quality evaluation by the measurement apparatus 1 of the frozen tuna loaded by a user. By viewing the result display monitor 2, the user may confirm the quality evaluation of the frozen tuna.

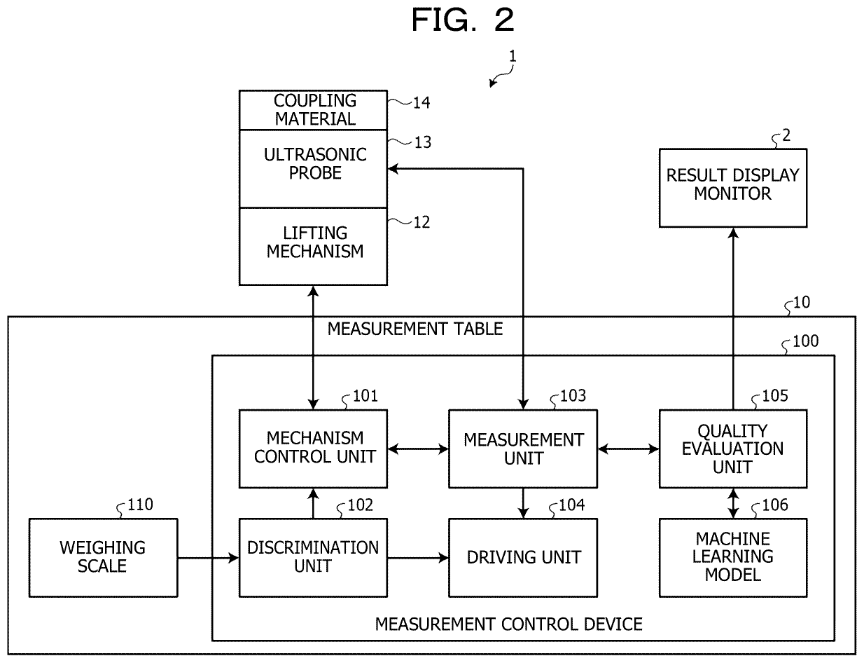

FIG. 2 is a block diagram of the measurement apparatus. In the measurement apparatus 1 of the present embodiment, a measurement control device 100 and a weighing scale 110 are installed inside the measurement table 10. However, the measurement control device 100 may be installed outside of the measurement table 10.

The weighing scale 110 measures a weight of frozen tuna in a state where the frozen tuna is placed in the recess 11 and supported and fixed by the stopper 16.

By using the ultrasonic probe 13, the measurement control device 100 measures the frozen tuna placed in the recess 11, and by using the obtained measurement data, the measurement control device 100 performs quality evaluation of the frozen tuna. Details of the measurement control device 100 will be described below. As illustrated in FIG. 2, the measurement control device 100 includes a mechanism control unit 101, a discrimination unit 102, a measurement unit 103, a driving unit 104, a quality evaluation unit 105, and a machine learning model 106.

The discrimination unit 102 has a weight threshold for discriminating that the frozen tuna is placed at an appropriate position in the recess 11. The weight threshold is preferably determined in accordance with the weight of the object to be handled. For example, the weight threshold is set by ranking for each fish or fish species. For example, in a case of the frozen albacore tuna, since the size is relatively small, the weight threshold is set to 10 kg, as an example. In a case of slightly large bigeye tuna called medium-sized bigeye tuna, the weight threshold is set to 20 kg as an example.

The discrimination unit 102 receives an input of the measurement result of the weight of the frozen tuna placed in the recess 11 by the weighing scale 110. When the measurement result is greater than or equal to the weight threshold, the discrimination unit 102 discriminates that the frozen tuna is placed in the recess 11. When a state where a fluctuation width of a numerical value of the measurement result greater than or equal to the weight threshold falls within a predetermined range continues for a certain period of time or longer, the discrimination unit 102 discriminates that frozen tuna is stationary. The predetermined range of the fluctuation width of the numerical value may be, for example, several grams. When the discrimination unit 102 discriminates that the frozen tuna is placed in the recess 11 and is stationary, the discrimination unit 102 instructs the mechanism control unit 101 to drive the lifting mechanism 12.

When the measurement result by the weighing scale 110 becomes less than the weight threshold after the frozen tuna is stationary, the discrimination unit 102 discriminates that the frozen tuna has been moved from the measurement table 10. The discrimination unit 102 notifies the driving unit 104 of the movement of the frozen tuna. In the present embodiment, although the discrimination unit 102 uses the same weight threshold for both the discrimination of placement and the discrimination of movement of the frozen tuna, for example, a value smaller than the weight threshold may be used as the threshold for the discrimination of movement. By using a value close to 0 as the threshold, for example, the discrimination unit 102 may reliably discriminate that the frozen tuna has been moved from the measurement table 10.

The mechanism control unit 101 controls extension and contraction of the lifting mechanism 12. When receiving instruction to drive the lifting mechanism 12 from the discrimination unit 102, the mechanism control unit 101 extends the lifting mechanism 12 and causes the ultrasonic probe 13 with the coupling material 14 attached to protrude from the opening of the recess 11. By extending the lifting mechanism 12 to a predetermined length, the mechanism control unit 101 causes the ultrasonic probe 13 with the coupling material 14 attached to be pressed against the frozen tuna, and causes the coupling material 14 to come into close contact with the frozen tuna due to the weight of the frozen tuna. Thereafter, the mechanism control unit 101 instructs the measurement unit 103 to start measurement.

When the measurement is completed, the mechanism control unit 101 receives a notification of the completion of the measurement from the measurement unit 103. By contracting the lifting mechanism 12, the mechanism control unit 101 separates the coupling material 14 from the frozen tuna, and houses the ultrasonic probe 13 with the coupling material 14 attached inside the measurement table 10.

The measurement unit 103 receives the instruction to start measurement from the mechanism control unit 101. By transmitting the information on the irradiation settings of the ultrasonic waves such as the frequency, the power, and the direction to the ultrasonic probe 13, the measurement unit 103 causes the ultrasonic probe 13 to irradiate the frozen tuna with ultrasonic waves, and acquires a reflected signal of the ultrasonic waves propagated through frozen tuna and received by the ultrasonic probe 13. With the measurement apparatus 1 according to the present embodiment, since frozen fish such as frozen tuna is measured, the measurement unit 103 causes the ultrasonic probe 13 to output ultrasonic waves in a low-frequency band from 20 kHz to 1 MHz.

The measurement unit 103 outputs measurement data indicating the reflected waves of the ultrasonic waves from the frozen tuna to the quality evaluation unit 105. The measurement unit 103 instructs the driving unit 104 to unlock the stopper 16.

Upon receiving the input of the measurement data indicating a waveform of the reflected waves of the ultrasonic waves with which the object is irradiated, the machine learning model 106 outputs an evaluation result indicating whether quality of the object is good or poor. For example, with respect to frozen tuna for which a correct answer of good or poor quality is found, the machine learning model 106 labels the correct answer of the quality evaluation result, and performs training by using learning data created in combination with the measurement data obtained from that frozen tuna.

The quality evaluation unit 105 receives the input of the measurement data from the measurement unit 103. Next, the quality evaluation unit 105 inputs the acquired measurement data to the machine learning model 106. Thereafter, the quality evaluation unit 105 acquires the quality evaluation result output from the machine learning model 106. The quality evaluation unit 105 causes the result display monitor 2 to display the quality measurement result of the frozen tuna placed on the measurement table 10. By referring to the evaluation result displayed on the result display monitor 2, the user may confirm the quality of the frozen tuna placed on the measurement table 10.

The driving unit 104 receives the instruction to unlock the stopper 16 from the measurement unit 103. The driving unit 104 separates the stopper 16 to release the fixation of the frozen tuna. When receiving a notification of movement of the frozen tuna from the discrimination unit 102, the driving unit 104 causes the stopper 16 to be fitted again to form an annular shape.

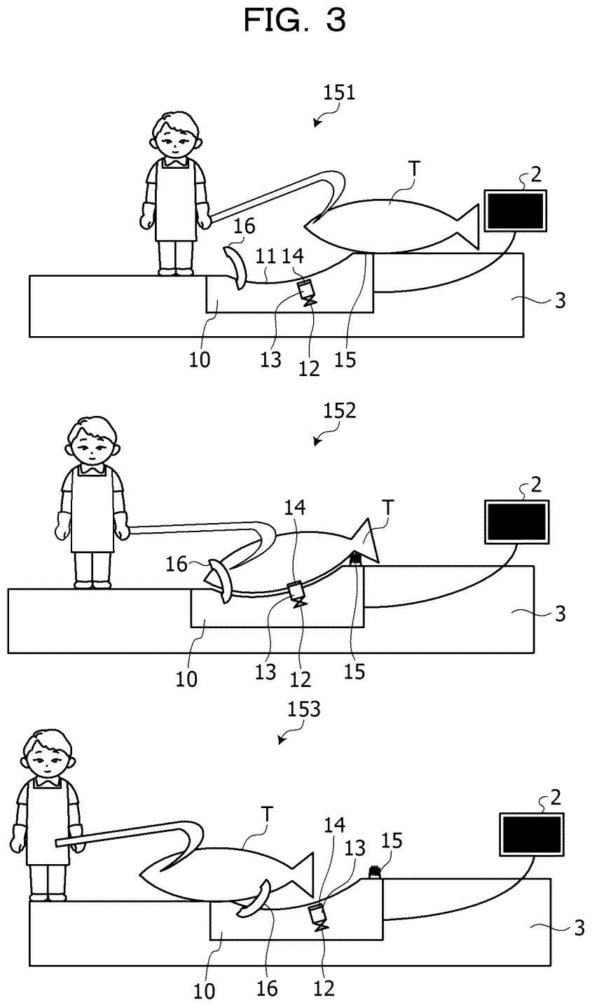

FIG. 3 is an image diagram of a method for using the measurement apparatus. A state 151 indicates a state before measurement, a state 152 indicates a state during measurement, and a state 153 indicates a state after measurement.

As illustrated in the state 151, the user pulls frozen tuna T from a high position on the ground 3 and carries the frozen tuna T to the high-side end of the inclined surface of the measurement table 10. The user pulls down the frozen tuna T toward the recess 11 provided in the inclined surface of the measurement table 10. At this time, dirt and ice blocks attached to a surface of the frozen tuna T are removed by the brush 15, and the surface is cleaned.

As illustrated in the state 152, a head of the frozen tuna T is inserted into the stopper 16, and the frozen tuna T is placed at a predetermined position in the recess 11. When the measurement control device 100 determines that the frozen tuna T is placed and is stationary based on a measurement result by the weighing scale 110 in the state 152, the measurement control device 100 lifts the lifting mechanism 12 and presses the coupling material 14 against the frozen tuna T to bring the coupling material 14 into close contact with the frozen tuna T. Next, the measurement control device 100 causes the ultrasonic probe 13 to irradiate the frozen tuna T with ultrasonic waves, causes the ultrasonic probe 13 to receive reflected waves of the ultrasonic waves to perform measurement, and acquires measurement data. Thereafter, the measurement control device 100 evaluates quality of the frozen tuna T based on the measurement data, and causes the result display monitor 2 to display the evaluation result. Thereafter, the measurement control device 100 unlocks the stopper 16.

After unlocking of the stopper 16, as illustrated in the state 153, the user pulls out the frozen tuna T from the recess 11 of the measurement table 10 and moves the frozen tuna T to the outside of the measurement table 10. By referring to the result display monitor 2, the user may confirm the quality measurement result of the frozen tuna T.

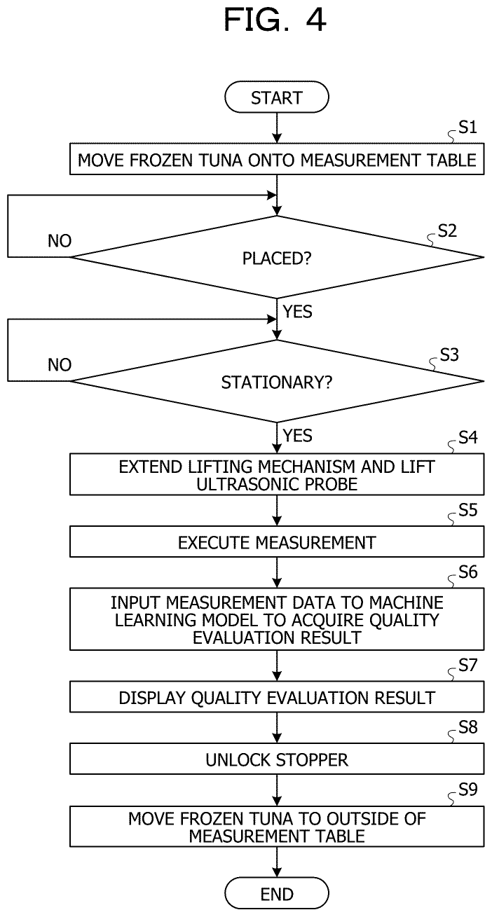

FIG. 4 is a flowchart of measurement and quality evaluation processing by the measurement apparatus according to the first embodiment. With reference to FIG. 4, a flow of the measurement and quality evaluation processing by the measurement apparatus 1 according to the first embodiment will be described.

A user moves frozen tuna onto the measurement table 10 (step S1). With a head inserted into the stopper 16, the frozen tuna is placed at a predetermined position in the recess 11.

By using a measurement result by the weighing scale 110, the discrimination unit 102 discriminates whether the frozen tuna is placed (step S2). For example, when the measurement result by the weighing scale 110 is less than a weight threshold, the discrimination unit 102 discriminates that the frozen tuna is not placed, and when the measurement result by the weighing scale 110 is greater than or equal to the weight threshold, the discrimination unit 102 discriminates that the frozen tuna is placed. When it is discriminated that the frozen tuna is not placed (step S2: No), the discrimination unit 102 waits until the frozen tuna is placed.

In contrast, when it is discriminated that the frozen tuna is placed (step S2: Yes), the discrimination unit 102 discriminates whether the frozen tuna is stationary (step S3). For example, when a state where a fluctuation width of the measurement result falls within a predetermined range continues for a certain period of time or longer, the discrimination unit 102 discriminates that the frozen tuna is stationary. When the frozen tuna is not stationary (step S3: No), the discrimination unit 102 waits until the frozen tuna becomes stationary.

In contrast, when the frozen tuna is stationary (step S3: Yes), the discrimination unit 102 instructs the mechanism control unit 101 to drive the lifting mechanism 12. When receiving the instruction to drive the lifting mechanism 12, the mechanism control unit 101 extends the lifting mechanism 12 to a predetermined length and lifts the ultrasonic probe 13 with the coupling material 14 attached (step S4). The ultrasonic probe 13 with the coupling material 14 attached protrudes from the opening of the recess 11, the coupling material 14 is pressed against the frozen tuna, and the coupling material 14 comes into close contact with the frozen tuna due to the weight of the frozen tuna. Thereafter, the mechanism control unit 101 instructs the measurement unit 103 to start measurement.

When receiving the instruction to start measurement, the measurement unit 103 causes the ultrasonic probe 13 to irradiate the frozen tuna with ultrasonic waves and receive reflected waves from the frozen tuna, and executes measurement (step S5).

Next, the measurement unit 103 outputs measurement data to the quality evaluation unit 105. The quality evaluation unit 105 inputs the measurement data to the machine learning model 106. Thereafter, the quality evaluation unit 105 acquires a quality evaluation result output from the machine learning model 106 (step S6).

Next, the quality evaluation unit 105 causes the result display monitor 2 to display a quality measurement result of the frozen tuna placed on the measurement table 10 (step S7). By referring to the evaluation result displayed on the result display monitor 2, the user may confirm the quality of the frozen tuna placed on the measurement table 10.

When receiving the instruction to unlock the stopper 16 from the measurement unit 103, the driving unit 104 separates the stopper 16 and unlocks for the frozen tuna (step S8).

When unlocking is performed for the frozen tuna, the user pulls out the frozen tuna from the recess 11 and moves the frozen tuna to the outside of the measurement table 10 (step S9). When the discrimination unit 102 discriminates that the frozen tuna has been moved by using the measurement result of the weighing scale 110, the driving unit 104 causes the stopper 16 to be fitted again.

While a mechanism in which one opening is provided in the recess 11 and the ultrasonic probe 13 protrudes from the opening is provided in the present embodiment, a mechanism in which a plurality of openings are provided in the recess 11 and the ultrasonic probe 13 protrudes from each opening may be provided. In this case, the measurement apparatus 1 may perform measurement by transmitting and receiving ultrasonic waves at a plurality of points of the frozen tuna, and may perform quality evaluation by using respective pieces of measurement data.

As described above, with the measurement apparatus according to the present embodiment, when frozen fish is pulled and placed in a recess on a measurement table, a head portion of the fish is fixed by a stopper. When the measurement apparatus discriminates that the fish is placed and stationary based on a measurement result of a weighing scale, the measurement apparatus causes a ultrasonic probe to which a coupling material is attached to protrude from an opening of the recess and causes the coupling material to come into close contact with the fish due to the weight of the fish. By using the ultrasonic probe, the measurement apparatus measures the fish by ultrasonic waves, and by using measurement data, executes quality evaluation of the fish.

By placing the fish in the recess and fixing the fish by the stopper in this manner, the fish may be placed at a predetermined position, and the measurement may be executed at an appropriate position. By lifting up the ultrasonic probe from below and bringing the ultrasonic probe into close contact with the tuna by the weight of the tuna, the ultrasonic probe may be pressed against the tuna with a sufficient force, and the adhesion may be improved. Consequently, it is possible to perform measurement in a state where the ultrasonic probe is pressed against and brought into close contact with the tuna at the appropriate position by applying the sufficient force, and it is possible to perform accurate data measurement in which fluctuation is suppressed. By using the measurement data with a high accuracy, it is possible to improve an accuracy of the quality evaluation.

By using the coupling material, a degree of adhesion may be further increased, and the accuracy of data measurement and the accuracy of the quality evaluation may be improved. By sliding the frozen fish along the inclined recess while passing the frozen fish over a brush, it is possible to remove attached substances such as dirt and ice blocks and improve the accuracy of the data measurement and the accuracy of the quality evaluation. By performing the measurement after the frozen fish that is the object becomes stationary, the ultrasonic probe may be pressed against the frozen fish at the appropriate position, and the accuracy of the data measurement and the accuracy of the quality evaluation may be improved. In the related art, a work of an operator for determining the appropriate position and pressing the ultrasonic probe against the position occurs every time, which takes time for each inspection, but with the measurement apparatus according to the present embodiment, the frozen fish is automatically disposed at a predetermined position and the ultrasonic probe is automatically pressed against the frozen fish, and thus it is possible to reduce an inspection time.

### First Modification Example

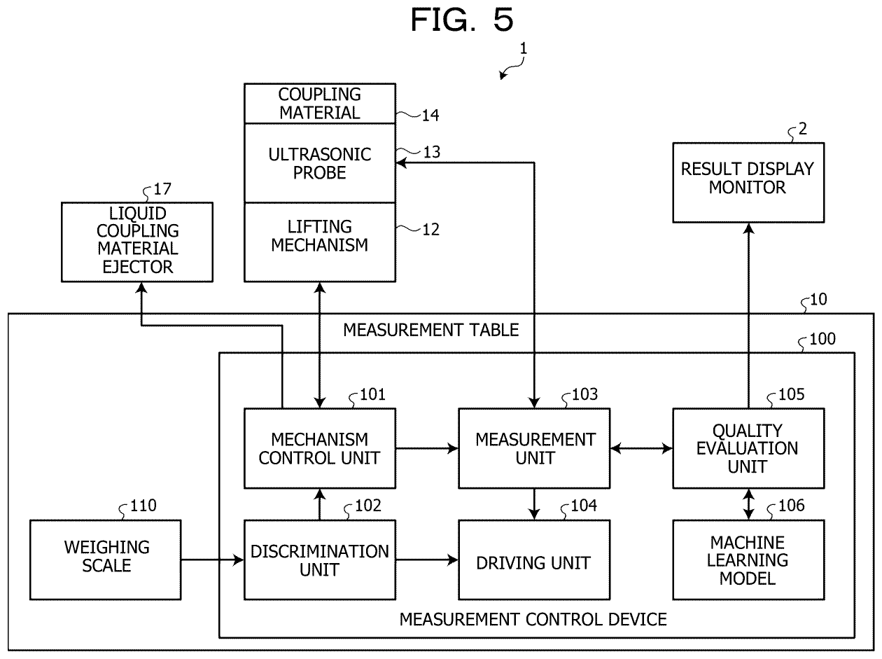

FIG. 5 is a block diagram of a measurement apparatus according to a first modification example. A measurement apparatus 1 according to the first modification example further includes a liquid coupling material ejector 17.

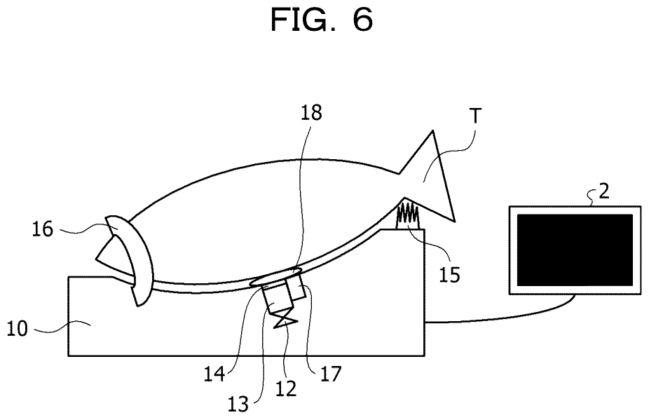

The liquid coupling material ejector 17 holds a liquid coupling material, which is a liquid medium for increasing adhesion to the frozen tuna. In the first modification example, the liquid coupling material ejector 17 holds glycerin as the liquid coupling material. FIG. 6 is a schematic diagram illustrating a mechanism of the measurement apparatus according to the first modification example. As illustrated in FIG. 6, the liquid coupling material ejector 17 is installed adjacently in an upper portion of the inclination of the measurement table 10 with respect to the ultrasonic probe 13 with the coupling material 14 attached.

When receiving an instruction to eject the liquid coupling material from the mechanism control unit 101, the liquid coupling material ejector 17 ejects glycerin from the recess 11 side. The ejected glycerin flows along the inclined surface of the recess 11 and forms a layer 18 covering a surface of the coupling material 14 that comes into contact with the frozen tuna. As described above, the liquid coupling material ejector 17 corresponds to an example of a “medium ejection unit” and ejects glycerin, which is a medium, between the ultrasonic probe 13, which is a data acquisition unit, and the frozen tuna, which is an object.

The description is continued by returning to FIG. 5. When the discrimination unit 102 discriminates that the frozen tuna is placed in the recess 11 and stationary, the mechanism control unit 101 instructs the liquid coupling material ejector 17 to eject the liquid coupling material. Thereafter, the mechanism control unit 101 extends the lifting mechanism 12 to press the ultrasonic probe 13 with the coupling material 14 attached having the glycerin-coated layer 18 formed on the contact surface with the frozen tuna, against the frozen tuna.

As described above, in the measurement apparatus according to the first modification example, glycerin is applied to the contact surface of the coupling material mounted on the ultrasonic probe with the frozen tuna, and the ultrasonic probe is pressed against the frozen tuna. Since glycerin is applied to the coupling material, the adhesion to the frozen tuna is further improved. Consequently, it is possible to further suppress the reflection of the ultrasonic waves on the contact surface of the frozen tuna, improve the accuracy of the data measurement, and improve the accuracy of the quality evaluation.

### Second Modification Example

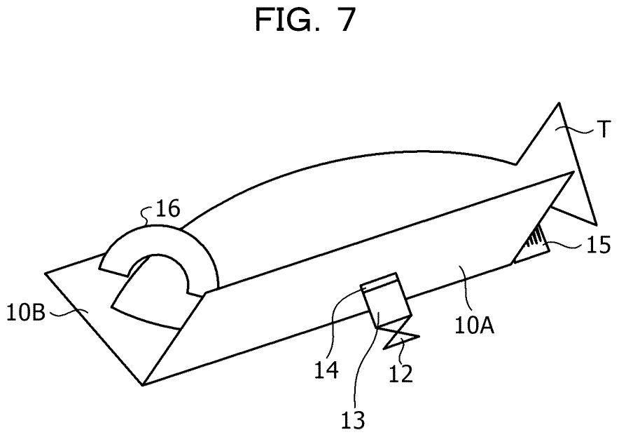

FIG. 7 is a schematic perspective diagram of a measurement apparatus according to a second modification example in a state where frozen tuna is placed. A measurement apparatus 1 according to the second modification example has a V-shaped groove having inclined surfaces 10A and 10B facing each other on the inclined surface. Both of the inclined surfaces 10A and 10B have recesses 11 in which the placed frozen tuna is accommodated. In a state where the frozen tuna is placed on the measurement table 10 and fixed to the stopper 16, the frozen tuna is placed in contact with both the recesses 11 of the inclined surfaces 10A and 10B.

Each of the recesses 11 of the inclined surfaces 10A and 10B has an opening, and the ultrasonic probe 13 with the coupling material 14 attached that protrudes from the opening and the lifting mechanism 12 are installed in each of the recesses 11. While the ultrasonic probe 13 with the coupling material 14 attached and the lifting mechanism 12 on the inclined surface 10A side are illustrated in FIG. 7, the similar ultrasonic probe 13 with the coupling material 14 attached and the similar lifting mechanism 12 are also provided on the inclined surface 10B side.

When the discrimination unit 102 discriminates that the frozen tuna is placed in the recesses 11 and stationary, the mechanism control unit 101 extends the lifting mechanisms 12 on both the inclined surface 10A side and the inclined surface 10B side to achieve close contact with the frozen tuna at two measurement sites.

By causing both of the ultrasonic probes 13 to transmit and receive ultrasonic waves, the measurement unit 103 executes measurement. The measurement unit 103 acquires measurement data of the frozen tuna at the two measurement sites.

By using the measurement data of the frozen tuna at the two measurement sites for the machine learning model 106, the quality evaluation unit 105 performs quality evaluation.

While the groove having the two inclined surfaces 10A and 10B is formed in the inclined surface of the measurement table 10 and the ultrasonic waves are transmitted and received at the two measurement sites in the present modification example, the configuration is not limited thereto. For example, the measurement apparatus 1 may acquire measurement data by performing transmission and reception of ultrasonic waves at each position in a state where a groove having three or more inclined surfaces is formed in the inclined surface of the measurement table 10 and the ultrasonic probe 13 is disposed on each surface, and may perform quality evaluation by using the measurement data.

As described above, the measurement apparatus according to the second modification example may perform measurement at different measurement sites of frozen tuna, and may perform quality evaluation by using measurement data at the plurality of measurement sites. By increasing the number of measurement sites in this manner, the possibility of acquiring accurate measurement data is improved, and the accuracy of the quality evaluation may be improved.

### Third Modification Example

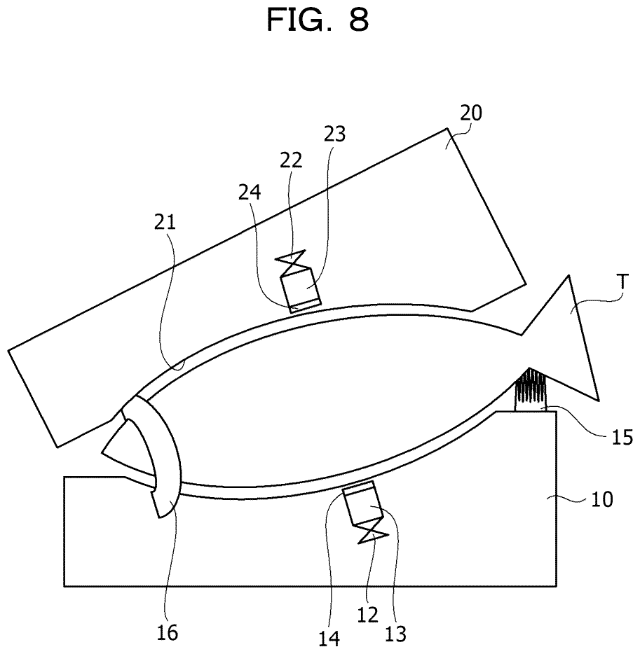

FIG. 8 is an image diagram of a measurement apparatus according to a third modification example in a state where frozen tuna is placed. A measurement apparatus 1 according to the third modification example includes a lid 20 that sandwiches frozen tuna from a position facing the measurement table 10. The lid 20 has a mechanism for opening and closing.

A recess 21 in which the frozen tuna is accommodated when the frozen tuna is sandwiched is formed in the lid 20. An opening is provided in the recess 21. A lifting mechanism 22, an ultrasonic probe 23, and a coupling material 24 are installed in the lid 20 so as to be able to protrude from the opening. However, the ultrasonic probe 23 with the coupling material 24 attached on the lid 20 side may be normally in a state of protruding from the opening without being provided with the lifting mechanism 22.

When it is discriminated by the discrimination unit 102 that frozen tuna T is placed and stationary, the lid 20 is closed and the frozen tuna is sandwiched between the recess 11 and the recess 21. The lid 20 may be driven by the driving unit 104 or may be moved by an operator, for example. When the lid 20 is sufficiently heavy, the coupling material 24 is brought into close contact with the frozen tuna by the weight of the lid 20. When the lid 20 is not sufficiently heavy, the coupling material 24 may be brought into close contact with the frozen tuna by mechanically pressing the lid 20 from above.

Thereafter, the measurement unit 103 extends the lifting mechanisms 12 and 22 and presses the ultrasonic probe 13 with the coupling material 14 attached and the ultrasonic probe 23 with the coupling material 24 attached against different measurement sites of the frozen tuna.

By causing both the ultrasonic probes 13 and 23 to transmit and receive ultrasonic waves, the measurement unit 103 executes measurement. The measurement unit 103 acquires measurement data of the frozen tuna at the two measurement sites.

By using the measurement data of the frozen tuna at the two measurement sites for the machine learning model 106, the quality evaluation unit 105 performs quality evaluation.

With the measurement apparatus according to the third modification example, measurement may be performed at different measurement sites of the frozen tuna, and quality evaluation may be performed by using measurement data at the plurality of measurement sites. By increasing the number of measurement sites in this manner, the possibility of acquiring accurate measurement data is improved, and the accuracy of the quality evaluation may be improved.

While the measurement using the ultrasonic waves and the quality evaluation using the measurement data for the machine learning model have been described above, the method for acquiring the measurement data obtained by measuring the state of the object and the quality evaluation method are not limited thereto. For example, the measurement apparatus may perform measurement using X-rays or the like, or may perform simple quality evaluation using a threshold or the like.

### Second Embodiment

A second embodiment will now be described. To increase the accuracy of the quality evaluation, it is preferable to perform the measurement at a plurality of points of the frozen tuna. However, when a plurality of ultrasonic probes are uniformly pressed against the frozen tuna having an individual difference in shape with orientations of the ultrasonic probes fixed, it may be difficult to successfully perform the measurement depending on postures of the ultrasonic probes. Since states of surfaces of the frozen tuna at individual measurement sites are different from each other, the way of pressing each ultrasonic probe may be inappropriate. In the ultrasonic measurement, the quality of data to be obtained greatly varies depending on the way of pressing the ultrasonic probe, and thus data acquisition may be unstable with such a method.

A measurement apparatus 1 according to the second embodiment is also represented by the block diagram of FIG. 2. However, the measurement apparatus 1 according to the second embodiment rotatably holds each of the plurality of ultrasonic probes 13 with respect to the frozen tuna. By simultaneously pressing the plurality of ultrasonic probes 13 against different measurement sites of the frozen tuna, the measurement apparatus 1 according to the second embodiment executes measurement at each measurement site. In the following description, description of functions of individual units that are the same as those in the first embodiment may be omitted. Each of the ultrasonic probes 13 collectively pressed against the different measurement sites in the present embodiment corresponds to an example of an “individual position data acquisition unit”.

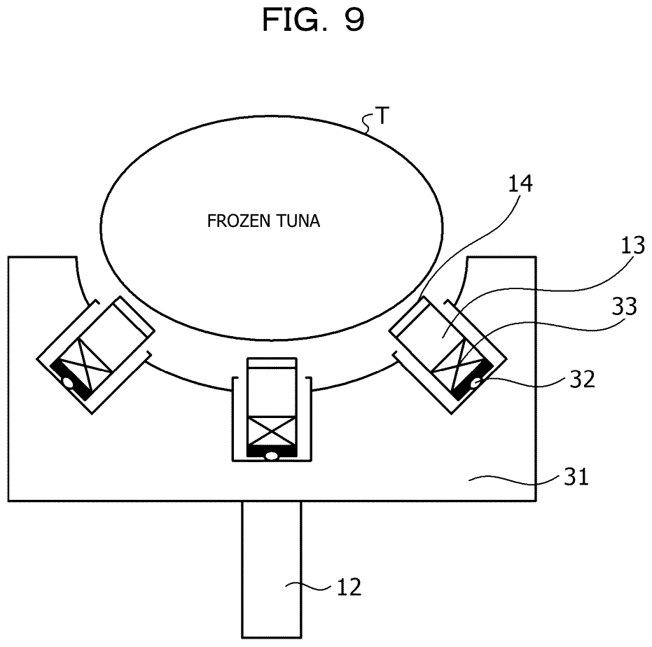

FIG. 9 is a schematic diagram of a plurality of ultrasonic probes for performing measurement of the frozen tuna. For example, the measurement apparatus 1 includes three sets of the ultrasonic probes 13 and the coupling materials 14 as illustrated in FIG. 9. In the present embodiment, a spring 33 is coupled to an end of the ultrasonic probe 13 on the measurement table 10 side. The spring 33 is rotatably fixed to a fixing member 31 forming a part of the recess 11 of the measurement table 10 in a state where a rotation angle is limited by a ball joint 32. The ball joint 32 has a predetermined rotation angle. For example, the ball joint 32 has a rotation angle of ±15 degrees.

With the ball joint 32, the ultrasonic probe 13 with the coupling material 14 attached may rotate within the predetermined rotation angle with respect to the frozen tuna T, and may be pressed against the surface of the frozen tuna T at an appropriate angle. For example, the ultrasonic probe 13 which is the individual position data acquisition unit comes into contact with the object with a degree of freedom of the predetermined rotation angle with respect to the object.

The recess 11 according to the present embodiment has an opening corresponding to the fixing member 31 to which the three ultrasonic probes 13 with the coupling material 14 attached is attached.

The lifting mechanism 12 is coupled to the fixing member 31 from the ground 3 side and supports the fixing member 31 from the ground 3 side. Under the control of the mechanism control unit 101, the lifting mechanism 12 extends an actuator after the frozen tuna becomes stationary. By using the actuator, the lifting mechanism 12 presses the fixing member 31 toward the frozen tuna in a state where the frozen tuna is placed in the recess 11, and causes each of the three ultrasonic probes 13 with the coupling material 14 attached to protrude from the opening.

By being pressed by the lifting mechanism 12, each of the ultrasonic probes 13 with the coupling material 14 attached is pressed against the frozen tuna T. At this time, inclinations and states of individual contact surfaces of the frozen tuna T against which the ultrasonic probes 13 with the coupling material 14 attached are pressed are different from each other. Each of the ultrasonic probes 13 with the coupling material 14 attached is independently rotated by the ball joint 32 in accordance with the inclination or state of each contact surface, and is individually pressed against the frozen tuna T in an appropriate direction.

Since the coupling material 14 is appropriately in close contact with the frozen tuna at each measurement site, the measurement unit 103 may accurately perform measurement at each point.

As described above, the measurement apparatus according to the second embodiment holds the ultrasonic probe so as to be rotatable within a predetermined rotation angle with respect to the frozen tuna. With this configuration, even when a plurality of ultrasonic probes are simultaneously pressed against the frozen tuna as the object, each of the ultrasonic probes may be pressed against the frozen tuna at an appropriate angle and may be brought into close contact with the frozen tuna. Consequently, it is possible to improve the accuracy of data measurement. By performing measurement at a plurality of points by using the plurality of ultrasonic probes, it is possible to improve the accuracy of the quality evaluation as a whole. Consequently, it is possible to improve the accuracy of individual measurement in each measurement site and to improve the accuracy of the quality evaluation as a whole.

### Third Embodiment

Next, a third embodiment will be described. As described in the second embodiment, when the probes are uniformly pressed against the frozen tuna having individual differences in shape, appropriate measurement may be difficult depending on the postures of the probes. For example, when a plurality of ultrasonic probes are used, the quality of data to be obtained greatly varies depending on the way of pressing the ultrasonic probes, and thus data acquisition may be unstable. Consequently, when the quality evaluation is performed by simply using the obtained measurement data, a prediction accuracy of the machine learning model may be significantly affected, and it may be difficult to perform appropriate quality evaluation.

A measurement apparatus 1 according to the third embodiment is also represented by the block diagram of FIG. 2. However, the measurement apparatus 1 according to the third embodiment includes a plurality of ultrasonic probes 13. In the following description, description of functions of individual units that are the same as those in the first embodiment may be omitted.

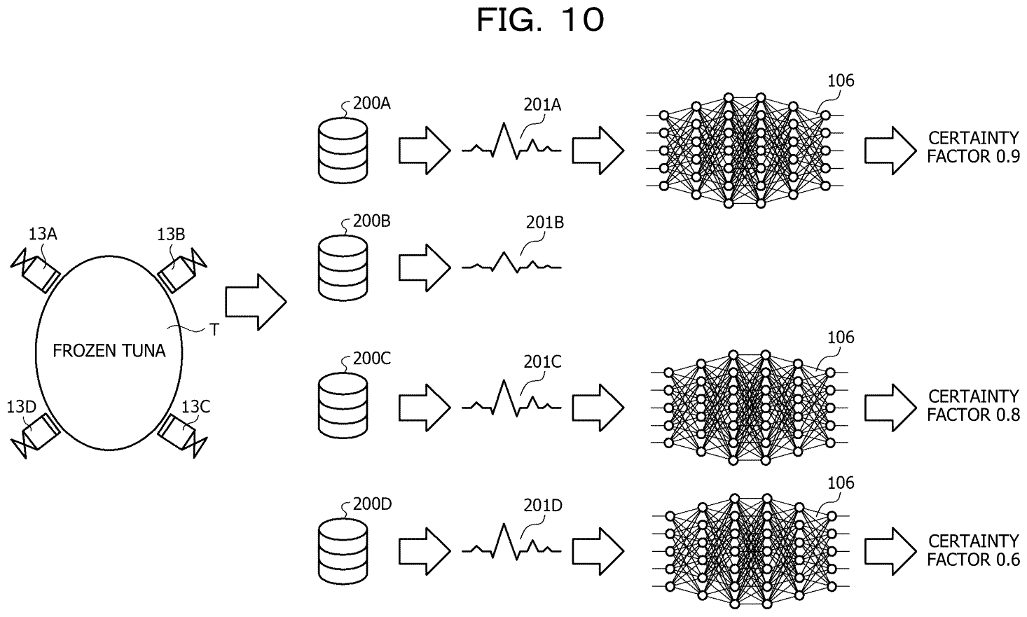

FIG. 10 is a diagram illustrating a procedure of quality evaluation by the measurement apparatus 1 according to the third embodiment. A case where measurement is performed by using four ultrasonic probes 13A to 13D will be described. For example, the measurement apparatus 1 including the ultrasonic probes 13A to 13D may be implemented by combining the second and third modification examples. Each of the ultrasonic probes 13A to 13D according to the present embodiment also correspond to an example of the “individual position data acquisition unit”.

By causing each of the ultrasonic probes 13A to 13D to transmit and receive ultrasonic waves to and from each measurement site of the frozen tuna T, the measurement unit 103 executes measurement at each measurement site. The measurement unit 103 outputs measurement data obtained at each measurement site to the quality evaluation unit 105.

When receiving instruction to re-execute the measurement from the quality evaluation unit 105, the measurement unit 103 causes each of the ultrasonic probes 13A to 13D to transmit and receive ultrasonic waves to and from each measurement site of the frozen tuna T again, and re-executes the measurement at each measurement site. In this case, a mechanism that shifts positions of the ultrasonic probes 13A to 13D may be provided in the measurement apparatus 1, and the measurement unit 103 may cause the mechanism control unit 101 to shift the positions of the ultrasonic probes 13A to 13D and then re-execute the measurement. Consequently, the contact states of the ultrasonic probes 13A to 13D with the coupling material 14 attached with the frozen tuna T change. In this case, when there is an ultrasonic probe from which appropriate measurement data is not obtained among the ultrasonic probes 13A to 13D, appropriate measurement data may be obtained by the re-execution.

The quality evaluation unit 105 has an exclusion threshold for excluding defective data for each measurement site. This exclusion threshold may be determined by using a machine learning model that determines whether the measurement data is good or poor, or may be determined by a person from statistical information or the like. With focus on an amplitude of the measurement data, the quality evaluation unit 105 uses a consistent value of the amplitude as the exclusion threshold in the present embodiment. With respect to the measurement sites corresponding to the ultrasonic probes 13A to 13D, the quality evaluation unit 105 has amplitudes 200A to 200D as the exclusion thresholds corresponding to the respective measurement sites.

The quality evaluation unit 105 has a certainty factor threshold for determining whether an evaluation result is usable in accordance with the certainty factor of the evaluation result. In the present embodiment, the certainty factor is represented by 0 to 1, and the quality evaluation unit 105 has 0.7 as the certainty factor threshold.

The quality evaluation unit 105 receives an input of the measurement data for each measurement site from the measurement unit 103. Next, the quality evaluation unit 105 determines whether measurement data obtained by the ultrasonic probe 13A is less than the amplitude 200A. When the measurement data obtained by the ultrasonic probe 13A is less than the amplitude 200A, the quality evaluation unit 105 excludes the data from inference data. The quality evaluation unit 105 determines whether measurement data obtained by the ultrasonic probe 13B is less than the amplitude 200B. When the measurement data obtained by the ultrasonic probe 13B is less than the amplitude 200B, the quality evaluation unit 105 excludes the data from the inference data. The quality evaluation unit 105 determines whether measurement data obtained by the ultrasonic probe 13C is less than the amplitude 200C. When the measurement data obtained by the ultrasonic probe 13C is less than the amplitude 200C, the quality evaluation unit 105 excludes the data from the inference data. The quality evaluation unit 105 determines whether measurement data obtained by the ultrasonic probe 13D is less than the amplitude 200D. When the measurement data obtained by the ultrasonic probe 13D is less than the amplitude 200D, the quality evaluation unit 105 excludes the data from the inference data.

For example, the quality evaluation unit 105 acquires measurement data 201A to 201D illustrated in FIG. 10 for the respective measurement sites. In this case, when the measurement data 201B is smaller than or equal to the exclusion threshold, the quality evaluation unit 105 excludes the measurement data 201B from the inference data. As described above, the quality evaluation unit 105 excludes the measurement data based on the exclusion threshold of the data quality from among the plurality of pieces of measurement data acquired by the ultrasonic probes 13A to 13D serving as the individual position data acquisition units to obtain the inference data.

Next, the quality evaluation unit 105 inputs the remaining measurement data to the machine learning model 106 as the inference data. The quality evaluation unit 105 acquires a quality evaluation result for each measurement site output from the machine learning model 106. In the present embodiment, the quality evaluation result has a certainty factor of 0 to 1. The quality evaluation unit 105 determines whether there is an evaluation result of less than 0.7, which is the certainty factor threshold, among the quality evaluation results for the respective measurement sites.

For example, the quality evaluation unit 105 uses the measurement data 201A, 201C, and 201D in FIG. 9 as the inference data, inputs them to the machine learning model 106, and acquires evaluation results of them. In this case, the quality evaluation unit 105 determines that the certainty factor of the evaluation result for the measurement site of the ultrasonic probe 13D is 0.6 and is less than the certainty factor threshold of 0.7.

When there is a measurement site for which the evaluation result is not obtained or when there is a measurement site for which the evaluation result with the certainty factor greater than or equal to the certainty factor threshold is not obtained, the quality evaluation unit 105 instructs the measurement unit 103 to re-execute the measurement. Until at least one evaluation result for which the certainty factor is greater than or equal to the certainty factor threshold is obtained for each measurement site, the quality evaluation unit 105 repeatedly causes the measurement unit 103 to execute the measurement and performs the quality evaluation. When an upper limit of the number of repetitions is set to a predetermined number of times and the number of repetitions reaches the upper limit, the quality evaluation unit 105 uses the evaluation result based on the measurement data obtained up to that point. With this configuration, the quality evaluation unit 105 repeats the processing of inputting each inference data to the machine learning model 106, acquiring a result that includes a certainty factor of quality evaluation for each inference data, causing each of the ultrasonic probes 13A to 13D to acquire measurement data based on the acquired certainty factor and a predetermined certainty factor threshold, and executing the quality evaluation.

Thereafter, for example, the quality evaluation unit 105 causes the result display monitor 2 to display an evaluation result with a highest certainty factor for each measurement site. Alternatively, the quality evaluation unit 105 may collect the evaluation results of the respective measurement sites to generate one evaluation result for the frozen tuna as the object, and may cause the result display monitor 2 to display the evaluation result.

As described above, the measurement apparatus according to the present embodiment executes measurement at a plurality of measurement sites, causes a machine learning model to perform quality evaluation by using inference data obtained by excluding measurement data smaller than or equal to an exclusion threshold, and repeats the measurement until evaluation results greater than or equal to a certainty factor threshold are obtained for the respective measurement site. With this, it is possible to perform data acquisition in consideration of an internal structure of frozen tuna for measurement data at a plurality of measurement sites, and it is possible to improve an accuracy of individual data measurement at each measurement site and improve the accuracy of the quality evaluation as a whole.

### (Hardware Configuration)

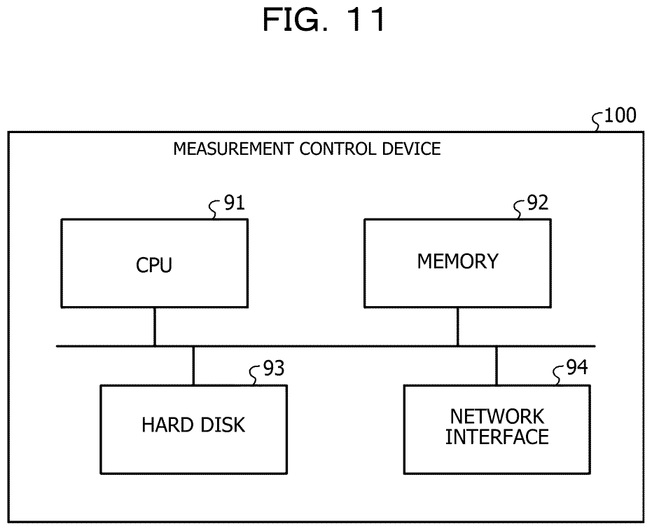

FIG. 11 is a hardware configuration diagram of the measurement control device. By referring to FIG. 11, an example of a hardware configuration for implementing each function of the measurement control device 100 will be described.

As illustrated in FIG. 11, the measurement control device 100 includes, for example, a central processing unit (CPU) 91, a memory 92, a hard disk 93, and a network interface 94. The CPU 91 is coupled to the memory 92, the hard disk 93, and the network interface 94 via a bus.

The network interface 94 is an interface for communication between the measurement control device 100 and an external device.

The hard disk 93 is an auxiliary storage device. The hard disk 93 stores the machine learning model 106 illustrated in FIGS. 2 and 5. The hard disk 93 stores various programs including programs for implementing the functions of the mechanism control unit 101, the discrimination unit 102, the measurement unit 103, the driving unit 104, and the quality evaluation unit 105 illustrated in FIGS. 2 and 5.

The memory 92 is a main storage device. For example, a dynamic random-access memory (DRAM) may be used as the memory 92.

The CPU 91 reads the various programs from the hard disk 93, loads the programs onto the memory 92, and executes the programs. With this, the CPU 91 implements the functions of the mechanism control unit 101, the discrimination unit 102, the measurement unit 103, the driving unit 104, and the quality evaluation unit 105 illustrated in FIGS. 2 and 5.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A measurement apparatus comprising:
a measurement table that has a recess inclined in a predetermined direction and an opening provided in the recess; and
a processor to:
discriminate whether an object is placed in the recess and is stationary;
protrude a data acquisition element from the opening when discriminating that the object is placed in the recess and is stationary;
press the data acquisition element against the object; and
acquire measurement data on the object from the data acquisition element.

2. The measurement apparatus according to claim 1, wherein
the processor acquires the measurement data by irradiating the object with ultrasonic waves.

3. The measurement apparatus according to claim 1, wherein
the measurement table includes a weighing scale that measures a weight of the object placed on the measurement table, and
the processor discriminates that the object is placed on the measurement table and is stationary based on a measurement result by the weighing scale.

4. The measurement apparatus according to claim 1, further comprising:
a liquid medium ejection element configured to eject a liquid medium, wherein
when discriminating that the object is placed in the recess and is stationary, the processor causes the liquid medium ejection element to eject the medium between the data acquisition element and the object, and causes the data acquisition element to protrude from the opening and presses the data acquisition element against the object with the medium interposed therebetween.

5. The measurement apparatus according to claim 1, wherein
the data acquisition element includes a plurality of individual position data acquisition elements that acquire the measurement data of different measurement sites in the object, and
each of the individual position data acquisition elements has a degree of freedom of a predetermined rotation angle with respect to the object and comes into contact with the object.

6. The measurement apparatus according to claim 1, wherein
the processor, as a machine learning model, receives the measurement data as an input outputs quality evaluation of the object; and
executes the quality evaluation of the object by using the machine learning model based on the measurement data.

7. The measurement apparatus according to claim 6, wherein
the data acquisition element
includes a plurality of individual position data acquisition elements that acquire the measurement data of the object at different measurement sites, and
the processor
repeats processing of excluding the measurement data based on an exclusion threshold of data quality from among a plurality of pieces of the measurement data to obtain inference data, inputting each of the inference data to the machine learning model to acquire a result that includes a certainty factor of the quality evaluation for each inference data, and causing each of the individual position data acquisition elements to acquire the measurement data based on the acquired certainty factor and a predetermined certainty factor threshold, and executing the quality evaluation.
